(19) United States
(12) Patent Application Publication
(10) Pub. No.: US 2005/0233400 A1
(43) Pub. Date: Oct. 20, 2005

(54) Proteomic method for predicting success of rescue cerclage

(76) Inventors: Carl P. Weiner, Towson, MD (US); Irina A. Buhimschi, New Haven, CT (US); Catalin S. Buhimschi, New Haven, CT (US)

Correspondence Address: R. Christopher Rueppell, Esq.;WHITEFORD TAYLOR & PRESTON, LLP, Seven St. Paul Street, Baltimore, MD, 21201-1626, US

(21) Appl. No.: 11/071,790

(22) Filed: Mar. 3, 2005

Related U.S. Application Data
(60) Provisional application No. 60/549,689, filed on Mar. 3, 2004.

(51) Int. Cl. G01N 33/53; G01N 33/537

(52) U.S. Cl. 435/7.92

(57) ABSTRACT

Through determination of the presence or absence of proteomic biomarkers within an amniotic environment, the present invention provides for the identification of the condition(s) within the amniotic environment and allows for predictive indication of the success of a pregnancy under the condition(s) and courses of treatment for the condition(s). The present invention further allows for a predictive indication of the success of a cerclage procedure performed on a cervix which supports the amniotic environment under the condition(s).

### CROSS REFERENCE TO RELATED APPLICATION

The instant application claims priority under 35 U.S.C. §119 to the U.S. Provisional Application Ser. No. 60/549,689, filed on Mar. 3, 2004, which is herein incorporated by reference in its entirety.

### FIELD OF THE INVENTION

The present invention generally relates to the field of reproductive science and more particularly to a method for determining the presence of a proteomic biomarker within an amniotic environment for predicting the success of a pregnancy and a particular course of treatment for preterm birth in general, and specifically an incompetent cervix.

### BACKGROUND

Spontaneous preterm birth is traditionally considered to be the result of spontaneous, idiopathic preterm labor or a functionally/structurally incompetent cervix. While causes of preterm birth and incompetent cervix have been identified there is currently no methodology which accurately and in a timely manner provides a predictive indicator of success of a pregnancy and/or course of treatment after the onset of these complications. Early midtrimester pregnancy loss associated with painless cervical dilation of undetermined etiology (i.e. incompetent cervix) remains a frustrating challenge. The traditional treatment of incompetent cervix is the placement of a purse string suture to close the internal cervical os. While typically placed electively in the early second trimester after a prior pregnancy loss deemed consistent with incompetent cervix. In this scenario, the risk of preterm birth approximates 30%, a figure essentially identical to the risk of recurrent spontaneous preterm labor. This suggests that the underlying causes of spontaneous preterm labor and delivery and incompetent cervix may be similar. The origin of incompetent cervix is likely multifactorial, and some pregnant women present already dilated and either no relevant or a confusing clinical history. In this setting, a cerclage can still be performed in an attempt to ‘rescue’ the pregnancy. The success of rescue cerclage performed for advanced cervical dilation absent labor is unpredictable. Exposure of the amniotic membranes to the vagina increases the risk of failure¹, and the reported failure rate of rescue cerclage is significantly higher than after elective placement^{2,3}.

Amniotic fluid microbial invasion is found in >50% of women with painless cervical dilation >2 cm between 14 and 24 w gestation.⁴ In one retrospective study, Mays et al noted 7/18 (39%) women considered candidates for rescue cerclage already had indirect markers of inflammation in their amniotic fluid.⁵ They declined to operate, and attributed their worse outcome to inflammation rather than the lack of surgery. However, a small randomized trial of rescue cerclage conducted by Althusius et al concluded rescue cerclage plus indomethacin was superior to bedrest alone.⁶ Thus, candidate selection for rescue cerclage remains an issue.

Cerclage failure manifests as either preterm labor with intact membranes, or labor preceded by preterm premature rupture of membranes (PPROM). While it is not unusual for preterm labor to be associated with PPROM, they are traditionally considered separate entities.⁷ At least four distinct patho-physiological pathways are implicated in the triggering of preterm birth⁸: stress, excessive stretching, decidual hemorhage and infection or inflammation.⁹

Decidual hemorrhage and the resulting placental abruption is recognized histologically in up to 60% of spontaneous preterm births, but is associated with recurrent vaginal bleeding in just 20%.⁸ Hemosiderin deposition is frequently observed, demonstrating the bleeding occurred at least 24-48 h antepartum. Further, deposition in the placental basal plate and extraplacental decidua is associated with lower mean birth weight percentiles in deliveries <32 w.^{10, 11}

There are multiple causes of inflammation, one being infection. Direct analysis of amniotic fluid is the most accurate technique for the detection of inflammation whether or not secondary to infection.^{12, 13} Tests used include amniotic fluid white blood cell and neutrophil counts, glucose concentration, Gram stain and microbial cultures.¹⁴ But even combinations of these tests have poor clinical performance¹⁵, and attempts to introduce them into practice have failed mostly because their results are not available in a clinically relevant time frame. As a result, diagnostic amniocentesis is usually not performed before placement of a rescue cerclage. It is, however, performed preoperatively to decompress the bulging amniotic sac and facilitate cerclage placement.

Proteomics provides a snapshot of all proteins present in a biological sample at a given moment. SELDI-TOF (surface-enhanced-laser-desorption-ionization-time-of-flight) technology combines chromatography with mass spectrometry¹⁶ and is commonly used for the identification of proteins within a biological sample. Such detection mechanisms have been used for the diagnosis of ovarian¹⁷ and breast cancers¹⁸, and Alzheimer's disease.¹⁹ Unfortunately, this type of detection and mechanisms for detection are not typically employed in the field of intra-uterine and/or intra-amniotic environment investigation, and have not been applied to women with incompetent cervix.

Therefore it would be desirable to provide a method for predicting the success of a pregnancy, course of treatment, and clinical success of rescue cerclage for an incompetent cervix and the possible need for additional therapies other than surgery utilizing the detection and analysis of a proteomic profile of the intra-amniotic environment.

### SUMMARY OF THE INVENTION

Accordingly, the present invention provides a method of identifying proteomic biomarkers within an amniotic environment which allow for a predictive indication as to the success of a pregnancy, particularly when cerclage is being contemplated and performed. The present invention further assists in the implementation of a course of treatment (i.e., treatment protocol) based upon the detection of the proteomic biomarkers which may increase the term of gestation. In a first aspect of the present invention, a method for identifying bleeding within an amniotic environment is provided. Through the identification of hemoglobins associated with the bleeding the underlying mechanism for the onset of pre-term labor, incompetent cervix, placental abruption, placenta previa, preterm labor, and/or identifying membrane rupture in the individual subject is allowed. Additionally, the present invention allows for the determination of as to whether the hemoglobin is fetal or maternal or truncated which. Fetal or maternal hemoglobin allow for identification of the source of the bleeding while the truncated hemoglobin allows for a determination of the age of the bleeding (old vs. recent).

In a second aspect of the present invention, a method for identifying inflammation within an amniotic environment supported by an incompetent cervix is provided. Proteomic biomarkers associated with an inflammatory state within the amniotic environment are identified and allow for the diagnosis of an underlying mechanism for pre-term labor, incompetent cervix, placental abruption, placenta previa, preterm labor, inflammation and infection.

In a third aspect of the present invention, the cervix is sutured (cerclage) and the determination of the absence or presence of the proteomic biomarker provides a predicitive indication of the clinical success of the cervical suturing in allowing the cervix to continue to support the amniotic environment, and where indicated the additon of therapies other than cerclage.

In a second aspect of the present invention, a method for determining the clinical success of a cerclage performed on a cervix supporting an amniotic environment includes determining if a proteomic biomarker is present within the amniotic environment supported by the cervix. Based upon this determination, a predictive indication of the clinical success of the cerclage may be diagnosed and used in counseling the patient on the predicted success of such surgical intervention.

### BRIEF DESCRIPTION OF THE DRAWINGS

FIG. 1 is a diagrammatic representation of protein profiles of amniotic fluid from five (5) patients undergoing rescue cerclage (cases: 1-5) and one (1) control patient (Cr1-6). (left: CHCA, right: SPA) The arrowheads indicate the peaks composing the MR score and representing Hb. (P1: neutrophil defensin-2: P2: neutrophil defensin-1: 3448.09 Da, P3: calgranulin C, P4: calgranulin A, P5 and P6: alpha and beta Hb chains). The MR score consist of protein markers of the innate immune system activation and was originally derived from studies of women in spontaneous preterm labor. In this scenario, the presence of 3 or more peaks was 100% predictive of failed labor inhibition. The x-axis of the tracings represents the molecular weight in kDa; the y-axis represents normalized peak intensity. R notes a reference protein peak present in all fluid samples. Phosphate buffer saline (PBS) is the sample diluent. The signal-to-noise (S/N) ratio from the PBS tracing is used to objectively estimate the presence or absence of a peak in the samples of amniotic fluid.

FIG. 2 is a diagrammatic representation of scattergrams illustrating the relationship between (A) latency (days amniocentesis-to-delivery), (B) percentage (%) prolongation pregnancy (latency divided by the GA (d) at amniocentesis×100) and MR score of the amniotic fluid samples obtained from patients before undergoing emergent cerclage (n=35). The two patients with documented intra-operative rupture of the membranes were excluded.

FIG. 3 is a diagrammatic illustration of representative SELDI tracings obtained with RBC lysates from 3 subjects.

FIG. 4 illustrates a series of ratios between the signal intensities of the gamma and beta or alpha chains.

FIG. 5 is an illustration of various hemoglobin chain peaks which allow for the identification of the origin of blood.

FIG. 6 is a diagrammatic representation of haemoglobin chain peaks including the peak of a carboxy terminal truncation of arginine.

FIG. 7 illustrates the mass shift between the alpha chain present in FIG. 6, the correlation to the terminal amino acid arginine in humans and the location of the arginine amino acid within the hemoglobin alpha chain of homo sapiens.

FIG. 8 is a diagrammatic representation of protein profiles providing a data source for distinguishing between recent versus past bleeding activity within an amniotic environment.

### DETAILED DESCRIPTION

The present invention applies proteomic technology to detect biomarkers indicative of the pathologic pathway activated to provide indications of pregnancy complications associated with preterm delivery such as intrauterine inflammation and decidual hemorhage in women. These complications may be further associated with other conditions, such as incompetent cervix. The Hb marker, which is found in the intra-amniotic environment as a result of hemorrhage, may also be applied to women in preterm labor. The determination of the presenece of the Hb marker in the amniotic environment provides a predictive indication of the success of the pregnancy, as desceribed in detail below. Further, inflammation or bleeding present in the amniotic fluid provides a predictive indication of cerclage success. Thus, the use of these proteomic biomarkers as predicitve indicators of the success of the pregnancy and clinical success of cerclage provides a significant advantage over the prior art. Further, these proteomic biomarkers allow for the development of treatment for complications experienced during pregnancy. These novel treatment options may assist in reducing the occurrence of pre-term delivery.

### SUMMARY OF STUDY

STUDY DESIGN: Amniocentesis was performed in thirty-seven (37) consecutive women to faciliate rescue cerclage. None had clinical evidence of chorioamnionitis. Thirty nine (39) unselected women undergoing genetic amniocentesis in the same unit during the same period served as control subjects. A proteomic fingerprint was generated by investigators blind to the clinical outcome. The MR score (0-4) was calculated to identify inflammation, and the peaks coresponding to hemoglobin chains (Hb) sought as an indicator of decidual hemorrhage or intra-amniotic bleeding.

RESULTS: Gestational age (GA) in the rescue cerclage group was 23.5 w [95% CI: 22.2-24.8 w] vs. 19.5 w [95% CI: 18.5-20.5 w] (p<0.01) in the control. The median cervical dilation at surgery was 4 cm [range: 2-9 cm]. Women undergoing cerclage delivered at 28.8 w [95% CI: 26.7-30.9 w], while control women delivered at 39.2 w [95% CI: 38.8-39.5 w]. Eighty seven percent of cerclage women delivered prematurely: 10 (32%)<24 w, and 22 (58%) between 24-37 w. Twenty-seven percent (10/37) of cerclage but no control subjects had a MR score indicative of intra-amniotic inflammation (MR=3-4) (p<0.001). Hb peaks were present in 11 (29.7%) cerclage but no control subjects. Outcome analysis was limited to the cerclage group. Two cases were excluded after intraoperative rupture of membranes. Clinical parameters included “latency” (GA delivery-GA amniocentesis) and the “% prolongation of pregnancy” ([latency/GA amniocentesis]×100). Rescue cerclage women with a MR=3-4 delivered earlier than those with a MR of 0-2 (p<0.001). Women with MR=3-4 had a shorter latency (median 3d, range: 0-105d) and a shorter % prolongation (median 1.8%, range 0-63%) compared to women with scores 0-2 (median latency 35d, range 0-148d, p<0.05; median % prolongation 17.9%, range 0-117%, p<0.05). Women with Hb had a shorter latency (median 6d, range: 0-100d) and a shorter % prolongation (median 3.8%, range 0-56.8%) compared to women without Hb (latency 38d, range 0-148d, p<0.05; median % prolongation 21.8%, range 0.7-116.5%, p<0.05). Hb was present in 70% (7/10) of women with a MR 3-4. Women with both a MR 3-4 and Hb (n=7, 19%) had the shortest intervals to delivery (median latency 3d, range 0-43d; median prolongation 1.9%, range 0-24%) (p<0.05 by survival analysis).

CONCLUSION: Thus, at least two pathologic mechanisms may be associated with incompetent cervix, those being an intrauterine inflammatory response and/or decidual hemmorhage. The existence of one or both of these pathologic mechanisms within the intra-aminiotic environment often predating surgery occurs in substantially half the women requiring rescue cerclage. Therefore, the activation of either mechanism may provide a predictive indication of cerclage failure. Preoperative diagnosis may allow pharmaceutical intervention to improve cerclage outcome. Further, while each pathway may function independently (i.e., only one pathway need be activated), their combined activation is synergistic. Thus, the incompetent cervix as described herein is not a reflection of structural cervical damage, but rather ill timed cervical ripening. Additionally, the same pathophysiologic mechanisms associated with spontaneous preterm labor and birth (inflammation and decidual hemorrhage) and its obligatory myometrial activation, can independently lead to cervical ripening absent myometrial activation.

### DETAILED DESCRIPTION AND DISCUSSION OF STUDY

A proteomic signature profile (termed the MR score) was described using the Ciphergen ProteinChip® that identified women with intrauterine inflammation secondary to chorioamnionitis without error.^{20, 21}. The description of such a proteomic signature profile is incorporated herein by reference. The MR score represents the arithmetic sum of Boolean indicators of absence (0) or presence (1) of four specific protein peaks as identified on a hydrophobic ProteinChip array. The four proteins peaks are identified as calgranulin A and C, and neutrophil defensins 1 and 2. The presence of three (3) or more in the amniotic fluid of women in preterm labor strongly predicts intra-amniotic inflammation identified using traditional criteria and imminent preterm delivery. The MR score is the first proteomic descriptor (biomarker) with demonstrated diagnostic value. The availability of such a highly accurate diagnostic tool could influence clinical management.

Preterm labor and incompetent cervix share pathophysiologic elements, and that either intra-amniotic inflammation as reflected in the MR score or intrauterine bleeding as identified by the presence of hemoglobin chains (Hb proteomic biomarker) occur in at least some women with incompetent cervix. Further, the success of rescue cerclage may reflect the absence of the neutrophil defensins, calgranulins and hemoglobin proteomic biomarkers from within the amniotic environment. This was confirmed in a consecutive series of women cared for at a single institution with cervical dilation in the second trimester secondary to incompetent cervix and no evidence of chorioamnionitis.

Materials and Methods

Cervical incompetence was diagnosed in 37 consecutive women with painless dilation (>2 cm) and no detectable uterine activity for a mean of 4 h [range: 1-24 h] prior to cerclage. In each instance, bulging but intact membranes were visualized at the level of the external cervical os. Each woman was a febrile and free of symptoms of chorioamnionitis at the time surgery. An emergency cerclage (McDonald type purse string) was performed under general anesthesia after amnioreduction to reduce the intra-amniotic fluid pressure and facilitate the cerclage procedure A cohort of 39 women with a sonographically normal pregnancy and cervix undergoing amniocentesis for chromosomal testing during the same study interval at the same clinical site provided the control amniotic fluid samples. All samples were centrifuged to remove cellular debris and stored at −80° C. They were collected under a protocol approved by the Institutional Review Board at the Hallym University School of Medicine and Kangnam Sacred Heart Hospital, Seoul, Korea, and all women provided written informed consent. Each amniocentesis and cerclage was performed by one investigator.

After delivery, the samples were coded by the investigator who performed each amniocentesis and cerclage, and transported to the remaining investigators without the key code. The proteomic fingerprint was generated, and the MR score (0-4) calculated and Hb peaks sought. The blinding was broken, and the relationship between the MR score, Hb and clinical outcome of the rescue cerclage assessed.

Protein Profiling Protocol for SELDI-TOF Mass Spectrometry:

The technique employed was previously described.^{20, 21} Briefly, 2 μl of 10-fold diluted amniotic fluid in phosphate buffer saline (PBS) was placed on spots of duplicate H4 arrays (8-spot H4 array, Ciphergen Biosystems, Fremont, Calif.). It is to be understood that other methodologies for the detection of the proteomic biomarkers may be employed, such as a Matrix Assisted Laser Desorption Ionization—Time of Flight (MALDI-TOF), without departing from the scope and spirit of the present invention. Additionally, methodologies which include immunoassays in various formats (e.g., Enzyme Linked Immunosorbent Assay (ELISA)) or a specific chemical reaction based on the structure of the specific marker may also be employed. Some spots were covered with PBS alone (five (5) spots for the entire experiment). After a one hour (1 h) incubation in a humidified chamber, the sample was aspirated and the spots washed individually with 25% aqueous acetonitrile solution, air-dried, and covered with matrix solution of either 1 μl of 20% saturated α-cyano-4-hydroxycinnamic acid (CHCA) in 25% trifluroacetic acid (TFA)/50% acetonitrile (ACN) on one array, or two sequential applications of 0.5 μl saturated solution (in 0,5% TFA/50% ACN) of sinapinic acid (SPA) on the other.

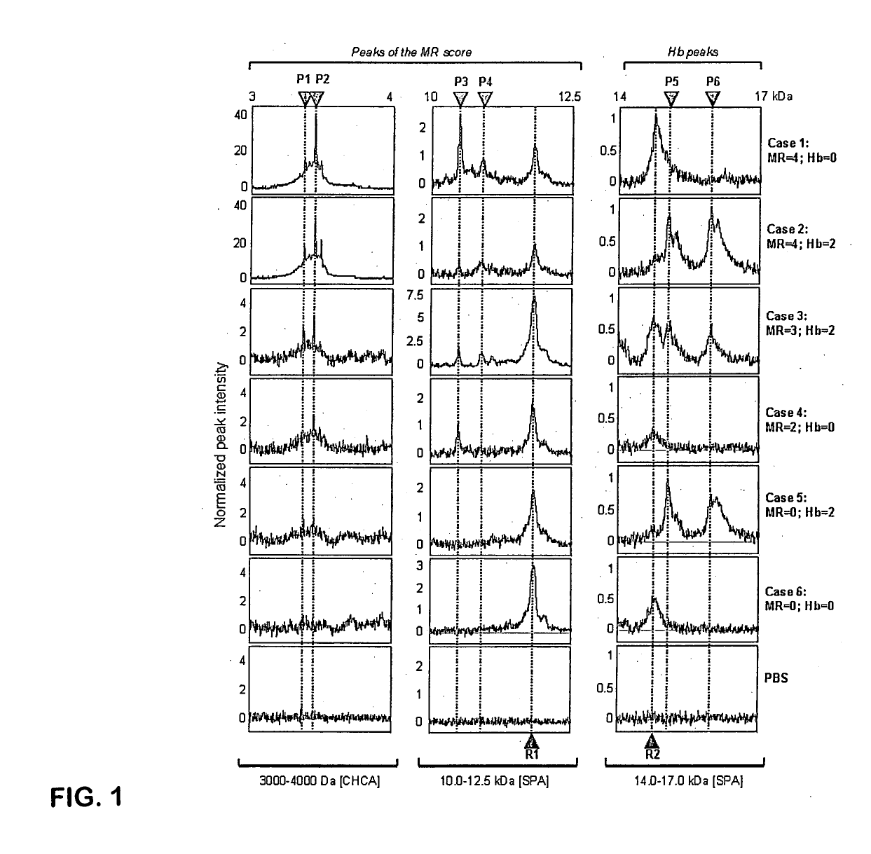

The arrays were read in the ProteinChip® Reader (Model PBS II) (Ciphergen Biosystems) using the ProteinChip® Software 3.0. SELDI tracings were examined in three mass ranges: 3000-4000 Da (CHCA), 10-12.5 kDa (SPA), 14-17 kDa (SPA). The data for the MR score peaks (P1-P4, FIG. 1) was extracted from the 3000-4000 Da (CHCA: P1 and P2), 10-12.5 kDa (SPA: P3 and P4). The data for the Hb peaks was analysed in the 14-17 kDa region (SPA: P5 and P6, FIG. 1).

Calculation of the MR and Hb Scores:

The presence or absence of a peak was determined objectively from numeric data exported into a spreadsheet using an algorithm that defines a peak based on the relative difference in signal-to-noise ratio (S/N) compared to the tracings obtained from the PBS covered spots at the corresponding mass value. A peak was considered present and assigned a value of one (1) if its S/N exceeded the average S/N +2SD from PBS spots (n=5) at the corresponding mass.

The MR score was originally derived by non-hierarchical analyses of SELDI tracings of amniotic fluid from women with and without intra-amniotic inflammation due to bacterial infection, and a data-mining algorithm based on the sequential application of five (5) Boolean logic criteria²¹. In the present study, the peaks composing the MR score were identified by their conspicuous aspect at, or in proximity to their known respective masses: 3377.01 and 3448.9 on the CHCA tracing (corresponding to neutrophil defensin-2 and -1, respectively) and at 10443.85 and 10834.51 Da (corresponding to calgranulin C and A, respectively).

The reported SwissProt masses of the alpha, beta and delta hemoglobin chains are 15126.36, 15867.22 and 15924.29 Da, respectively. Analogous to the MR score, presence or absence of the Hb peaks was objectively based on the S/N ratio on the PBS tracings. A value of one (1) was given for each Hb peak present.

Statistical Analysis:

Because the surgery was conducted across a range of gestational ages, the data was normalized to reflect the degree a pregnancy was prolonged after surgery. First, the “latency” interval was calculated, defined as the days gestation at delivery minus the days gestation at amniocentesis. Second, the “% prolongation of pregnancy” was calculated, which is defined as the latency divided by the days gestation at amniocentesis×100. It is to be understood that this calculation slightly underestimates the benefit of cerclage since the standard gestational age based on last menstrual period includes two weeks at the beginning when the pregnancy does not yet exist. Statistical analyses included testing for normality using the Kolmogorov-Smimov test, the Student T-test, Mann Whitney test, Kruskal Wallis ANOVA on ranks followed by Dunn's tests and survival analysis as appropriate. Comparisons between proportions were performed using the Fisher's exact test.

Clinical characteristics of the study groups are summarized in Table I. The average gestation at amniocentesis in the rescue cerclage group was 23.5 w (95% CI: 22.2-24.8 w) compared to 19.5 w (95% CI: 18.5-20.5 w) in the control group (p<0.001, t-test). The median cervical dilation at cerclage was 4 cm (range: 2-9 cm). Eighty seven percent of women in the rescue cerclage group delivered preterm: 10 (27%) below 24 w, and 22 (58%) between 24-37 w. Cervical dilation at cerclage was inversely correlated to both outcome measures (latency: r=−0.398, p<0.02; % prolongation of pregnancy: r=−378, p=0.02).

Women in the rescue cerclage group delivered at an average gestation of 28.8 w (95% CI: 26.7-39.6 w) with a median latency of 21d (range 0-148d). Identifiable postoperative complications related to preterm delivery were: intraoperative iatrogenic rupture of membranes (n=2), late abortion (n=3), preterm contractions with intact membranes (n=11), fever (n=2), spontaneous PPROM (with or without preterm contractions: n=9) or abruptio placenta (n=1).

In contrast to the cerclage group, all women in the control group delivered at term with a mean gestation of 39.2 w (95% CI: 38.9-39.6) and a median latency of 143d (range 56-170d).

SELDI Tracing Analysis:

Representative SELDI tracings from five (5) patients treated by rescue cerclage and one control patient undergoing genetic amniocentesis are illustrated in FIG. 1. The baseline noise level on the respective CHCA and SPA SELDI readings is illustrated by the tracing that included PBS instead of amniotic fluid. The peaks of the MR score (P1-P4) and those corresponding to the Hb chains (P5 and P6) are shown at their expected masses. Two additional, conspicuous peaks were present in the mass regions of interest in the majority of tracings (reference peaks: R1 and R2). The R1 peak, as previously identified, is a fragment of beta-2 microglobulin 20. Its characheristic SELDI peak was used for rapid orientation to the mass axis of the SPA spectra. The R1 peak was present in all fluids studied. The R2 peak was less conspicuous than the R1 peak under these experimental conditions, and was present on only thirty-four (34) of the tracings (67.5% of cases and 23% of controls). Although these proportions are different (p<0.01, Fisher's exact test), the R2 peak does not meet the criteria for a disease biomarker (all biomarker peaks must be absent in control subjects).^{20, 21} Table II illustrates the average experimental masses of the biomarker peaks with Boolean indicators of one (1) observed in the 76 samples of amniotic fluid included in this study.

Twenty-seven percent (10/37) of rescue cerclage but no control patients had a MR score of 3-4 (p<0.001, Fisher's exact). Twelve women in cerclage group had Hb peaks present (Hb score=2). Women with Hb present each showed both the alpha and beta peaks (Hb=2). No control patient sample had Hb peaks. Seven women had both a MR=3-4 and Hb score=2. Thus, almost half the women had an abnormal study.

Excluding the two women with accidental intraoperative rupture of membranes, cerclage women with an MR 3-4 delivered earlier than those with a MR=0-2 (p<0.05, Mann Whitney). Women with MR 3-4 had a shorter latency (median 3d, range 0-105d) and a shorter % prolongation (median 1.8%, range 0-62.8%) compared to women with scores of 0-2 (latency 35d, range 0-148d, p<0.04 Mann Whitney; median % prolongation 17.9%, range 0-116.5%, p<0.03 Mann Whitney).

Women with Hb peaks delivered earlier that those without (latency: Hb=0: median 38d, range 0-148d vs Hb=2: median 6d, range 0-100d, p<0.04; % prolongation: Hb=0: median 21.8%, range 0.7-116.5% vs Hb=2: median 3.8%, range 0-56.8, p=0.03 Mann Whitney). Thirty percent (3/10) women with a MR=3-4 had no Hb, while 42% (5/12) with Hb had a MR<3 (p=NS).

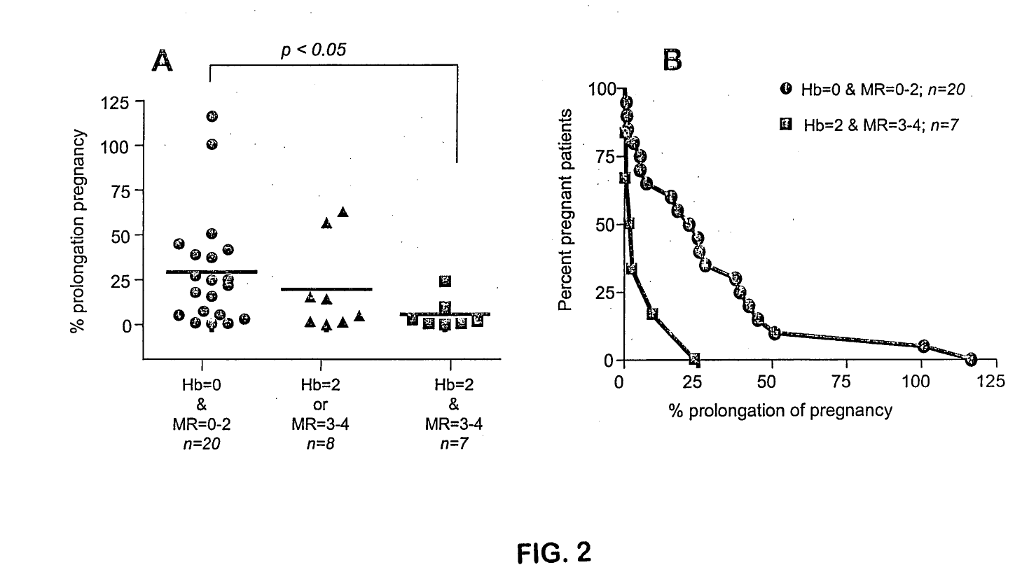

The combination of the MR score and Hb is a predictive indicator of cerclage success. Women with a MR<3 and no Hb had a median latency of forty and one-half days (40.5 d) (range 1-148 d) compared to those with either a MR=3-4 or Hb=2 (but not both) whose median latency was fourteen days (14 d) (range 0-105 d) compared to those with both a MR=3-4 and Hb=2 whose median latency was three days (3 d) (range 0-43 d). Likewise, the % prolongation among these three groups declined stepwise from 23.3% to 9.6% to 1.9% (Logrank Chi square 7.69, p=0.005, RR: 10.0 95% CI: 9.6-10.4]) (FIG. 2).

The causes of preterm delivery are likely multiple, and the clinical distinction between preterm labor and incompetent cervix often difficult. The present data teach the separation is artifical at least from the standpoint of the pathophysiologic mechanisms. Rescue cerclage is typically performed after the unexpected discovery of cervical dilation in the absence of detectable uterine activity prior to viability. It is long recognized that the success of rescue cerclage is relatively low, certainly compared to elective cerclage.^{2, 3} It is less clear how vaild this comparison is, and unknown whether losses occur despite the cerclage or because of it. Likely, the two are not mutually exclusive.

Based on the finidings outlined above the present invention demonstrates not only the existence of two potential pathophysiologic mechanisms associated with incompetent cervix that are also presumed causes of preterm labor, but also their novel value as predictive indicators of cerclage success. In almost half the women, evidence of either an intrauterine inflammatory response^{20, 21} or presumbably decidual hemorrhage²³ predating the rescue cerclage was found. Additionally, there is a stepwise worsening of outcome, failure of rescue cerclage and pre-term labor, when one or both of these pathways is active. In contrast, the absence of activity along either pathway evidences that the likelihood of cerclage success (prolonging of the gestation period for the fetus) is high, absent an operative complication.

Neutrophil defensins (alpha-defensins) belong to a family of cationic antimicrobial peptides. These key components of the host-defence mechanism²⁴ exert their bactericidal activity by punching pores into bacterial membranes.²⁵ Elevated defensin levels are found in ²⁶patients with sepsis, meningitis,²⁷ and cystic fibrosis.²⁸ Once thought the exclusive product of neutrophils²⁹, recent study indicates that alpha-defensins are also produced by CD8-T lymphocytes and may have HIV-1 suppressing activity.³⁰ Calgranulins are members of the S100 calcium-binding proteins³¹ implicated in Alzheimer's disease, cancer, cardiomyopathy, psoriasis, rheumatoid arthritis, and other inflammatory disorders. There are many causes of inflammation, and a determination of the specific cause of any inflammation found in the intra-amniotic environment may be clinically relevant.

Similar to inflammation, evidence of decidual hemorrhage is present in many women delivering after the spontaneous onset of preterm labor.^{23, 9} It is suggested the release of thrombin leads to myometrial activation and uterine contractions.^{32, 33} In the present series, a quarter of the women with an incompetent cervix had Hb in their amniotic fluid prior to the cerclage and absent detectable uterine contractions. Its uniform absence from the samples of control women strongly suggests that the Hb detected was not the result of the amniocentesis, but rather predated it. The observation that 30% of women with a high MR score had no Hb, and 42% with Hb had a normal MR score, reveals the two pathologic pathways have separate origins, but can intersect as they act additively, wherein thrombin reportedly stimulates matrix metalloproteinase expression in cultured endometrial stromal and decidual cells³⁴. Further, intracerebral hemorrhage is known to cause activation of both macrophages and matrix metalloproteinases³⁵ in the absence of bacterial infection.

The ability of the present invention to allow for the accurate and rapid identification of at risk women preoperatively creates an opportunity to intervene and improve outcome pharmacologically. There are several therapeutic possibilities. Antibiotics alone have failed to either improve cerclage results or prolong pregnancy in women with preterm labor and intact membranes. They have also failed to prevent delivery and maintain fetal health in animal models of preterm labor secondary to inflammation. Anti-inflammatory or antioxidant therapies may provide a pathophysiologically targeted therapy. For example, antioxidant treatment with N-acetyl cysteine (NAC) prevents preterm delivery and rescues the fetuses from inflammation-induced wastage in a model of preterm delivery secondary to E. coli lipopolysaccharide.³⁶ Treatment with an anti-inflammatory cytokine, interleukin-10, has a similar effect.³⁷ Thus, it may be possible to preoperatively treat with evidence of inflammation using a combination of NAC, or other anti-inflammatory agents perhaps coupled with a broad spectrum antibiotic agent that achieves high concentrations in the conceptus. Such treatment will require the rapid turn around provide by SELDI.

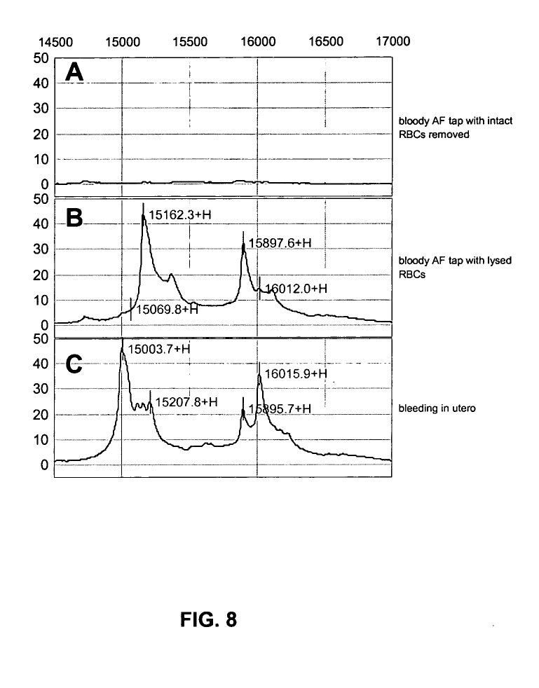

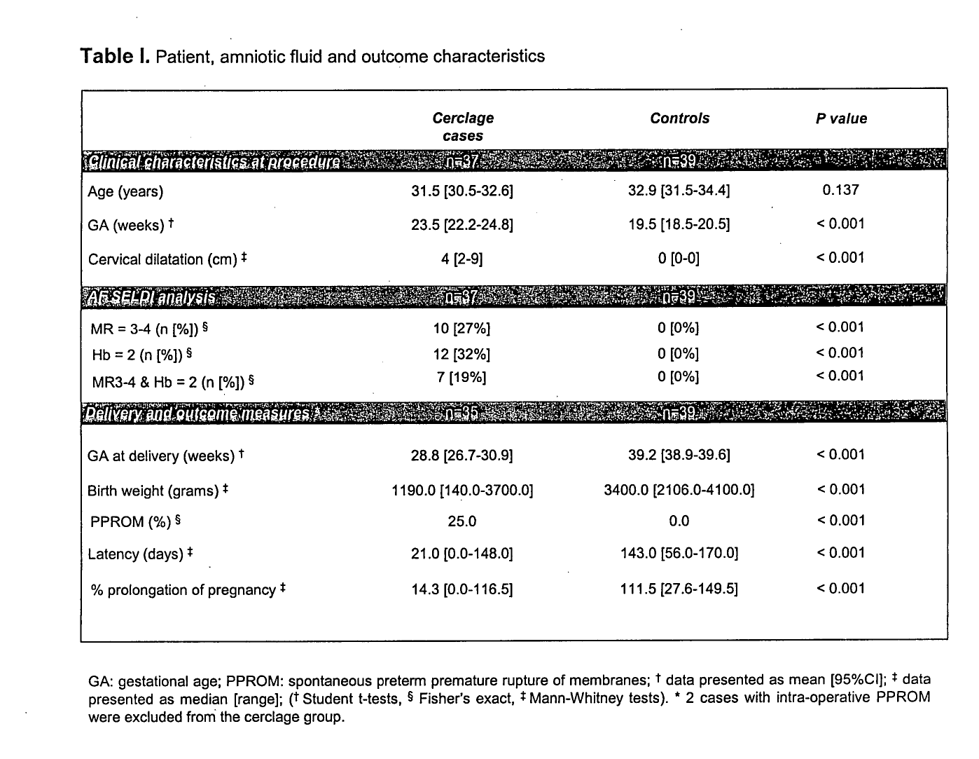

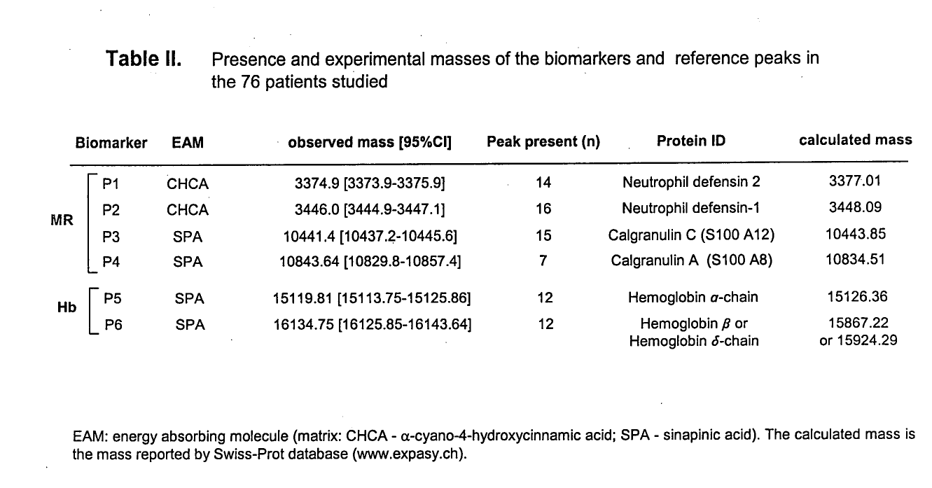

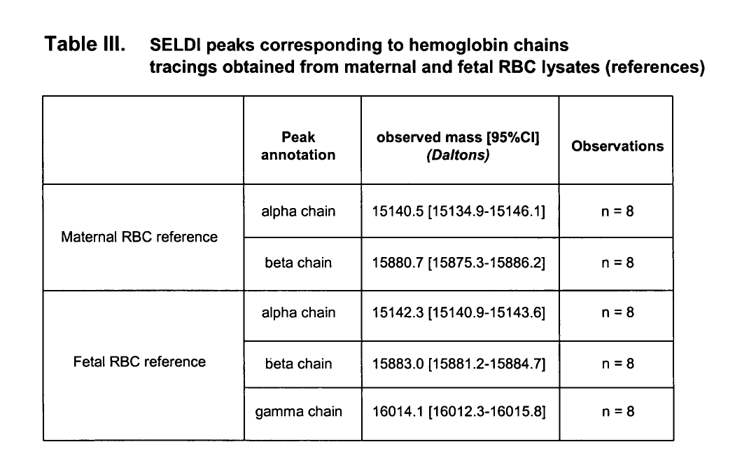

A method to distinguish between old and new bleeding (e.g. between a prior bleed into the amniotic cavity vs. a new bleed or a contamination occurring during amniocentesis, aka, bloody tap) may be provided. Referrring now to FIG. 8, tracing A shows a SELDI trace from a sample of amniotic fluid after a bloody tap after intact red blood cells were separated from the supernatant which is devoid of hemoglobin. Tracing B shows the same sample after freezing, thawing and centrifugation (i.e. fresh RBC that contaminate amniotic fluid are hemolyzed by the freeze thaw cycle and the contained hemoglobin released in the supernatant). Tracing C is from a patient that had in utero bleeding. There is a difference in aspect of the hemoglobin peak profile and the shift in molecular weight of the predominant peaks. For the alpha chain hemoglobin metabolism in vivo results in a decrease in molecular weight of approximately 150 Da. This change could be indicative of a bleeding that has occurred in vivo much earlier before the amniocentesis procedure as opposed to an acute bleeding or a traumatic tap. The aspect of the second group of peaks representing the beta chain in adults or beta and delta in fetuses is also modified in peak composition and aspect. This modification may discern between changes to adult versus fetal hemoglobin.

Significance of Bleeding for Preterm Delivery and Adverse Pregnancy Outcome:

Vaginal bleeding complicates almost a quarter of all pregnancies and is associated with doubling of the risk for preterm delivery.³⁸ About half of the bleeding episodes during pregnancy have unknown causes.^{39, 40} A study investigating the relation between self-reported vaginal bleeding during pregnancy and preterm birth in a prospective cohort of 2800 women in the US reported that multiple episodes and longer duration of bleeding is associated with both PPROM and preterm labor especially when occurring later in pregnancy.⁴¹ Some forms of placental dysfunction associated with vaginal bleeding are well established causes of preterm birth. Furthermore, the presence of hemosiderin deposition (a blood pigment catabolite indicative of old bleeding) in sections of placenta and fetal membranes are significantly associated with prematurity.⁴²

Whether the origin of perinatal bleeding is maternal or fetal remains controversial. Intra-amniotic hemorrhage during the ante partum or intra-partum is a life threatening emergency for the mother or fetus. While ante partum hemorrhage before twenty weeks (20 wks) gestation is frequently related to abortion/miscarriage (i.e. defective placental implantation), intra-partum hemorrhage most frequently results from placental abruption, placenta previa, invasive conditions of the placenta or fetal hemorrhage.⁴³ Currently, early detection of placental abruption relies on the observation of vaginal bleeding, or when overt bleeding does not occur, on tests such as the Kleihauer-Bethke stain or on the observation of the fetal heart rate tracing (sinusoidal pattern, decreased variability or sudden onset of fetal bradycardia).⁴⁴ An understanding of how to assess, plan, intervene and diagnose perinatal hemorrhage is important for prevention of perinatal tragedies that accompany hemorrhage in pregnancy.

Differentiating Between Maternal and Fetal Bleeding:

When vaginal bleeding is overt, the differential diagnosis between maternal and fetal bleeding can in theory be based on the alkaline denaturation tests (e.g. Apt test) which are based on the ability of fetal but not adult hemoglobin to resist alkaline denaturation. Another alternative is hemoglobin electrophoresis, but this procedure takes longer and requires laborious laboratory protocols. Intra-amniotic bleeding is frequently discovered incidentally at the time of amniocentesis performed to rule out genetic chromosomal abnormalities or infection/inflammation in women presenting either signs of symptoms chorioamnionitis or preterm birth. There is currently no standard means of determining whether the amniotic fluid contains either maternal or fetal blood. Prior study by G D Hankins demonstrated the simple discoloration of amniotic fluid (brown or green) at the time of genetic second-trimester amniocentesis as an isolated finding does not prognosticate a poor pregnancy outcome.⁴⁵ These findings seem at odds with our current observation that presence of free hemoglobin chains in amniotic fluid in women with incompetent cervix are associated with cerclage failure, but may be explained in the lack of specificity of the test used by Hankins (e.g nonspecific discoloration of amniotic fluid due to meconium).

SELDI Mass Spectrometry to Establish Origin of Fetal-Maternal Bleeding:

First we established reference SELDI peaks corresponding to maternal and fetal hemoglobin. For this aspect, paired maternal and umbilical venous blood samples were collected from eight (8) pregnancies. Ten (10) ul of packed RBC's were lysed in 1 ml distilled water. Lysates were spun and hemoglobin concentration in the supernatant was estimated using Drabkin's reaction (Stanbio). Three (3) microliters of a normalized solution containing 0.2 mg/dl hemoglobin was either dried onto spots of WCX2 ProteinCHIP arrays or incubated on either hydrophobic H4 arrays or immobilized metal affinity (IMAC3) overlaid with 100 uM ZnSO4. After 1 hour, the incubated arrays were washed with phosphate buffered saline (pH=7.4), air-dried and overlaid with two sequential applications of 50% saturated sinnapinic acid (SPA) in trifluoroacetic acid/50% acetonitrile.

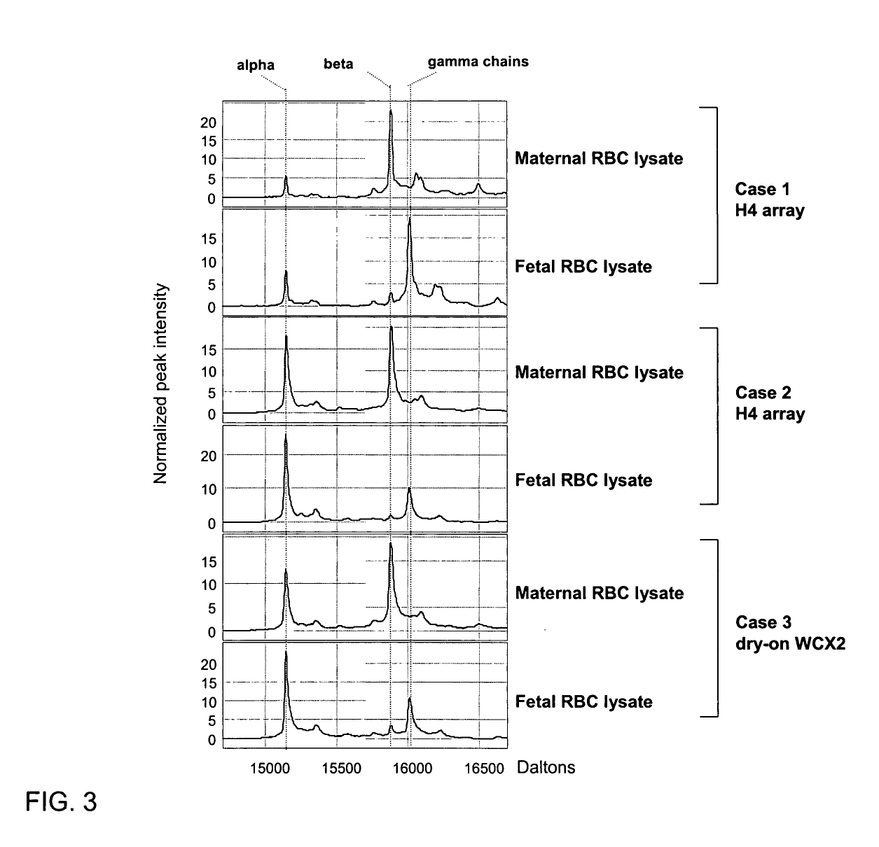

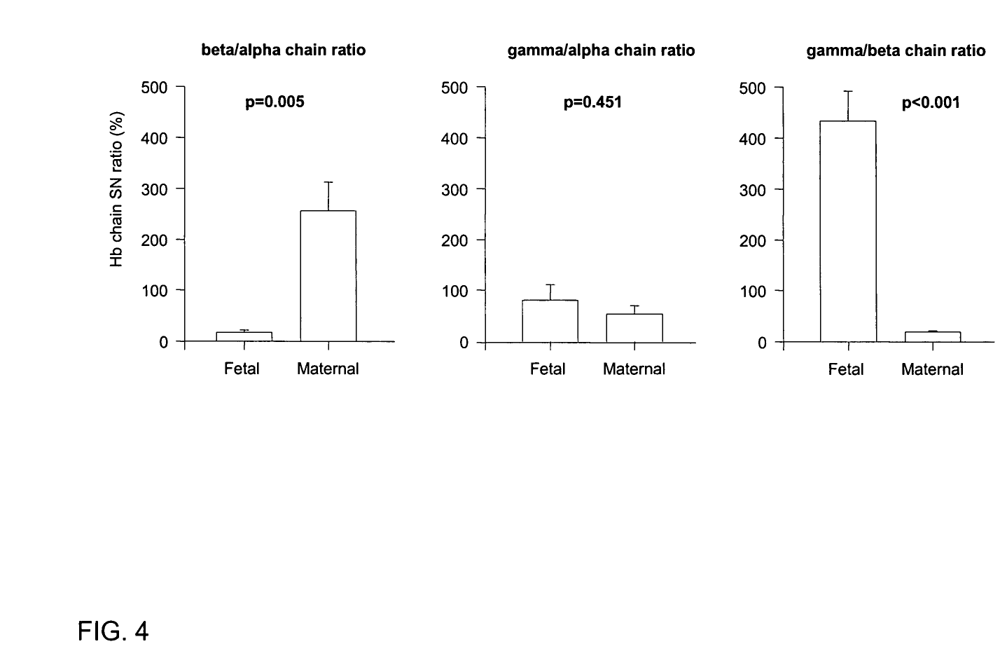

Referring now to FIG. 3, representative SELDI tracings were obtained with RBC lysates from three (3) subjects. The SELDI peaks corresponding to hemoglobin chains are found in the 14-17 kDa mass interval. After baseline subtraction and normalization for total ion current, several parameters for each peak—including peak intensity (height at centroid), signal-to-noise (S/N) ratio, mass (m/z−1) at centroid were exported to a computerized spreadsheet. A semi quantitative analysis was performed from the S/N ratio for each of the peaks represented in FIG. 3. The average masses of the peaks that correspond to the alpha, beta and gamma chains of human hemoglobin were identified from the maternal or respective fetal samples, respectively, and are presented in Table III. To render the peak analysis objective rather than subjective, we calculated a series of ratios between the signal intensities of the gamma and beta or alpha chains, as show in FIG. 4.

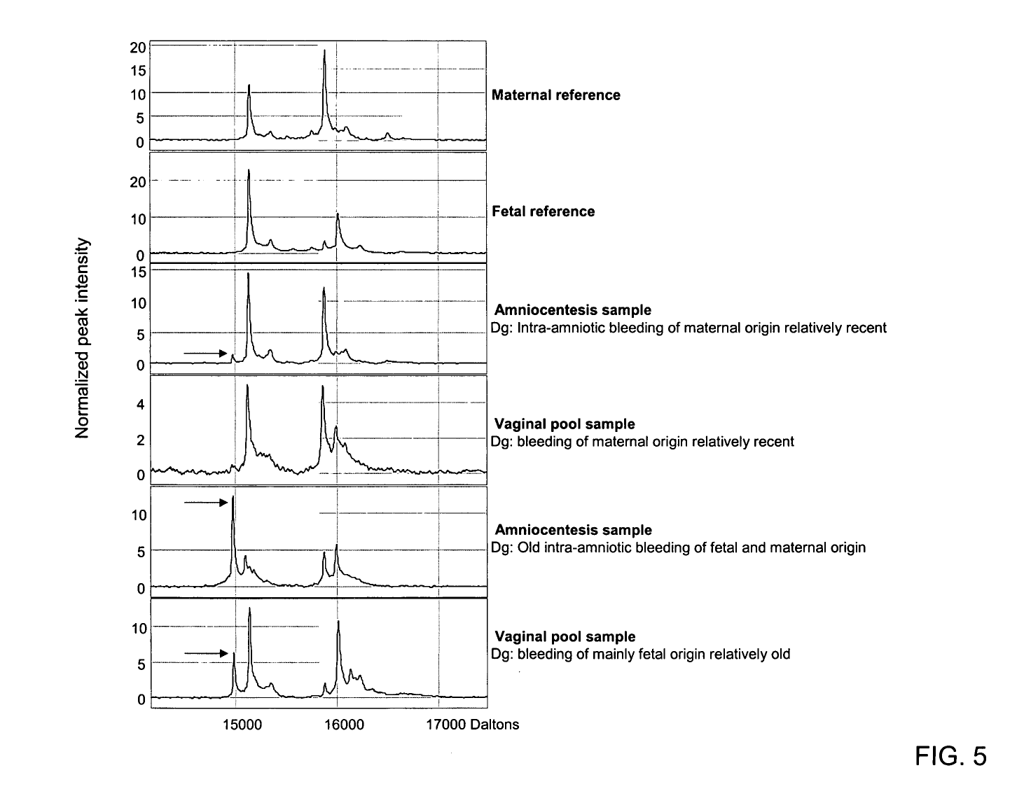

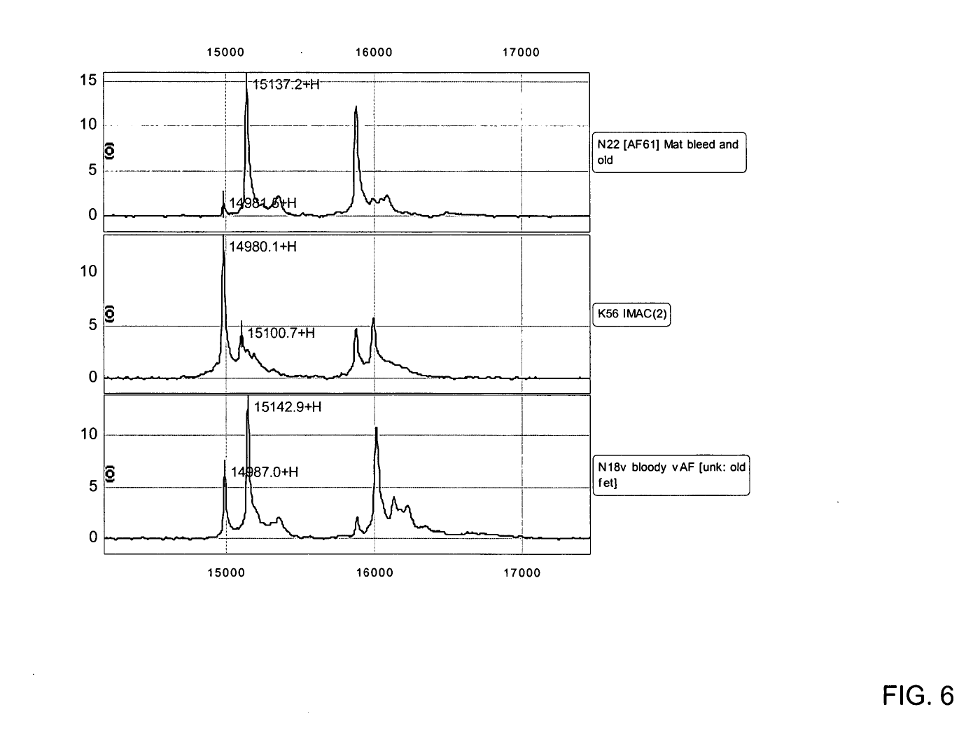

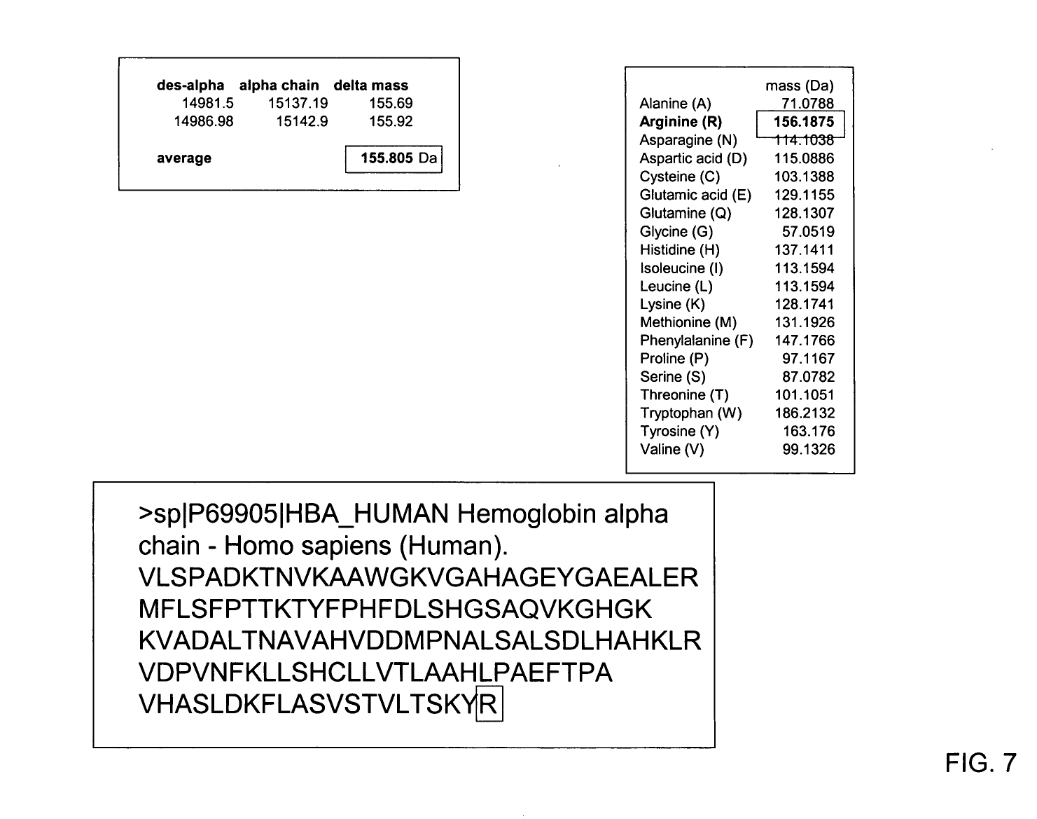

Next we examined a series of samples of amniotic fluid contaminated with blood of unknown origin obtained either by amniocentesis or from vagina in women with rupture of membranes. We were able to distinguish the peaks corresponding to hemoglobin chains, and by analogy, with the references described above to establish the origin of the blood loss, shown in FIG. 5. We also observed in some amniotic fluid samples with blood contamination that an additional peak with a mass lower than the alpha chain which was present especially in the samples that were darkly discolored and where the bleeding likely occurred at an earlier time (old bleeding).

SELDI Mass Spectrometry to Establish Old Versus Recent Bleeding in Amniotic Vaginal Fluid:

The IMAC-Zn arrays were superior identifying the lower molecular weight peak (des-alpha chain) which we suggest represents a catabolite (protein truncation) of the alpha chain. An analysis of the mass shift between the alpha chain present in the reference tracings revealed a difference in mass of approximately 156 Da. Given that the amino terminal amino acid in the human hemoglobin is arginine with a reported average mass of 156 Da, we could conclude that the peak identified in the respective discolored amniotic fluid samples (des-alpha chain) corresponded to a carboxy terminal truncation of the Arg141. The carboxy terminal amino acids in the beta and gamma chains are Histidine [His146]. The literature teaches that such carboxy terminal truncations of Arg141 are most probably the result of a carboxypeptidase enzymatic activity, 46 part of the endogenous catabolic process undergone by the free hemoglobin in amniotic cavity. Thus, the truncated des-Arg141 hemoglobin we find in samples either from amniocentesis or the vaginal pool is indicative of old bleeding into amniotic cavity. The presence of this peak can be easily identified at SELDI analysis on the IMAC3-Zn array.

The midtrimester loss of an otherwise healthy fetus after cervical dilation absent detectable uterine activity shares with preterm labor an association with intrauterine inflammation and presumed decidual hemorrhage. Though the phrase ‘cervical incompetence’ suggests a structural weakness or deficiency, the instant application demonstrates there is more to it than just physical strength. The simultaneous assessment of a number of distinct biomarkers using a convenient platform such as SELDI allows for the rapid and accurate identification of high risk women, creating the possibility for new therapeutic interventions to reduce the failure rate of rescue cerclage.

In a preferred embodiment, the present invention provides a method for identifying bleeding within an amniotic environment. The identification of bleeding is determined by the presence or absence of at least one hemoglobin chain (Hb) biomarker within a collected sample of the amniotic environment. The sample may be collected using various methodologies, such as amnicentesis or directly from the vagina. The Hb biomarker provides a basis upon which the success of a pregnancy may be predicted. Further, the presence of the Hb biomarker provides a predictive indication of various complications associated with a pregnancy, such as incompetent cervix, placental abruption, placenta previa, preterm labor, rupture of a fetal membrane, infection and inflammation.

The detection of the Hb biomarkers further allows for the determination of the source of the bleeding (fetal vs. maternal) within the amniotic environment, as described previously. This sourcing determination may provide valuable information for the treatment of bleeding complications experienced during pregnancy. The implementation of a course of treatment specifically tailored to the bleeding and directed at the appropriate source may assist in increasing the gestation period of the pregnancy. The detection of the Hb biomarkers also allow for the determination of the staleness of the bleeding, that is whether the bleeding occurred recently or in the past. As described above, identification of a truncated hemoglobin chain provides the indication of staleness of the bleeding which may provide further assistance in the development of a proper course of treatment. It is to be understood that the course(s) of treatment employed may include those previously identified or those which are contemplated by one of ordinary skill in the relevant art.

In another exemplary embodiment, the present invention provides a method for identifying inflammation proteomic markers within an amniotic environment. In the current embodiment, the relevant protein markers of inflammation within the amniotic environment include a cationic antimicrobial peptide, specifically a neutrophil defensin, and a calcium-binding protein, specifically calgranulin. The determination of the presence or absence of these proteomic biomarkers within the amniotic environment provide a basis for predictive indication of the success of a pregnancy and/or the success of a cerclage procedure in maintaining the pregnancy. The presence of these biomarkers may indicate various complications, such as incompetent cervix, placental abruption, placenta previa, preterm labor, rupture of a fetal membrane, infection and inflammation. Further, the determination of the presence of these biomarkers allows for the development of courses of treatment specifically tailored to the problem. For example, in an inflammatory environment the treatment options/protocols may include administration of various medications/compounds, such as anti-inflammatory compounds, antioxidant compounds, and/or antibiotics, as previously described. These treatment options may reduce the risk of complications during pregnancy and/or the effects of the complications.

In a preferred embodiment, the present invention provides a method for treatment of a cervix which is supporting an amniotic environment. Support of an amniotic environment being that circumstance where a women is pregnant and the amnioitic environment is established intra uterine. It is contemplated that the cervix may be in a non-dilated state or a dilated state and that the pregnancy may be pre-term (before the 37th week of gestation). It is further contemplated that a pre-term dilation of the cervix may be a painless dilation which is consistent with an incompetent cervix. Treatment of the cervix may begin with the determination of the presence or absence of proteomic biomarkers within the amniotic environment. As previously identified, these biomarkers may include neutrophil defensins, calgranulins and/or hemoglobins, which may be used as predicitive indicators. It is to be understoood that other proteomic signature profiles may be identified within the amniotic environment and serve as predictive indicators of the success of a cerclage procedure. After the presence or absence of the biomarkers is determined the cervix may be sutured (cerclage). The cerclage of the cervix may be an “elective” or “rescue” cerclage procedure, without departing from the scope and spirit of the present invention.

With the cervix sutured the clinical success of the cerclage procedure may be predicited by analysis of the biomarker information gathered. No indication of biomarkers based on the proteomic profile provides a strong predicitive indication that the cerclage will be successful. Success of the cerclage being measured by the amount of gestation time given the fetus after the cerclage procedure. The longer the gestation, such as to term, the more successful the cerclage is considered. Therefore, the absence of biomarkers in the amniotic environments strongly indicates a prolonged gestation time.

In amniotic environments where biomarkers are found, the success of a pregnancy or after cerclage which is measured by the gestation time, may be reduced. The biomarkers, as previously stated, provide an indication of inflammation or hemmorhaging within the amniotic environment. The presence of these biomarkers allows a predictive indication that the cerclage will have limited success. Further, the number of biomarkers present, such as a single biomarker versus a plurality of biomarkers, may have an additive impact upon the success of the cerclage. Limited success being evidenced by a non-term (less than 37 weeks) gestation time. As evidenced by the data from the study outlined above, the gestation time for pregnancy with such a contaminated amniotic environment may range from a few short hours, to day(s), to week(s).

In an alternative embodiment, the present invention provides a method for determining the clinical success of a cerclage procedure. It is contemplated that the cerclage procedure may be “elective” or “rescue” in nature. In the current embodiment the determination of the presence or absence of proteomic biomarkers within the amniotic environment provides the basis for the prognostic capabilities of the present invention. As stated above, the presence of these biomarkers provide a predictive indicator that the cerclage will be of limited success while the absence of such biomarkers provides a predictive indicator that the cerclage will be successful.

The present invention demonstrates not only the existence of two potential pathophysiologic mechanisms associated with pre-term labor and incompetent cervix, but also their novel value as predictive indicators of pregnancy and cerclage success. Thus, the present invention may provide a significant advantage in tailoring treatment options for pregnant women, particularly for those women who experience an incompetent cervix.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

### REFERENCES

¹ Yip S K, Fung H Y, Fung T Y. Emergency cervical cerclage: a study between duration of cerclage in situ with gestation at cerclage, herniation of forewater, and cervical dilation at presentation. Eur J Obstet Reprod Biol 1998; 78: 63-7.
² Charles D, Edwards W R. Infectious complications of cervical cerciage. Am J Obstet Gynecol 1981; 141:1065-71.
³ Wu M Y, Yang Y S, Huang S C, Lee T Y, Ho H N. Emergent and elective cerclage for cervical complications. Int J Gynaecol Obstet 1996; 54: 23-9.
⁴ Romero R, Gonzalez R, Sepulveda W, Brandt F, Ramirez M, Sorokin Y, Mazor M, Treadwell M C, Cotton D B. Infection and labor. Viii. Microbial invasion of the amniotic fluid cavity in patients with suspected cervical incompetence: Prevalence and clinical significance. Am J Obstet Gynecol 1992; 167: 1086-91.
⁵ Mays J K, Figueroa R, Shah J, Khakoo H, Kaminsky S, Tejani N. Amniocentesis for selection before rescue cerclage. Obstet Gynecol 2000; 95: 652-5.
⁶ Althuisius S M, Dekker G A, Hummel P, van Geijn H P; Cervical incompetence prevention randomized cerclage trial. Cervical incompetence prevention randomized cerclage trial: emergency cerclage with bed rest versus bed rest alone. Am J Obstet Gynecol 2003; 189: 907-10.
⁷ Blanco J D. Clinical problem of preterm labor. Clin Obstet Gynecol 2000; 43: 713-6.
⁸ Lockwood C J, Kuczynski E. Markers of risk for preterm delivery. J Perinat Med 1999; 27: 5-20.
⁹ Lockwood C J, Kuczynski E. Risk stratification and pathological mechanisms in preterm delivery. Paediatr Perinat Epidemiol 2001;15 Suppl 2:78-89.
¹⁰ Sherer D M, Salafia C M. Chronic intrauterine bleeding and fetal growth at less than 32 weeks of gestation. Gynecol Obstet Invest 2000; 50: 92-5.
¹¹ Salafia C M, Lopez-Zeno J A, Sherer D M, Whittington S S, Minior V K, Vintzileos A M. Histologic evidence of old intrauterine bleeding is more frequent in prematurity. Am J Obstet Gynecol 1995;173:1065-70.
¹² Yoon B H, Romero R, Kim C J, Jun J K, Gomez R, Choi J H, Syn H C. Amniotic fluid interleukin-6: a sensitive test for antenatal diagnosis of acute inflammatory lesions of preterm placenta and prediction of perinatal morbidity. Am J Obstet Gynecol 1995; 172: 960-70.
¹³ Angus S R, Segel S Y, Hsu C D, Locksmith G J, Clark P, Sammel M D, Macones G A, Strauss J F 3rd, Parry S. Amniotic fluid matrix metalloproteinase-8 indicates intra-amniotic infection. Am J Obstet Gynecol 2001; 185: 1232-8.
¹⁴ Romero R, Yoon B H, Mazor M, Gomez R, Diamond M P, Kenney J S, Ramirez M, Fidel P L, Sorokin Y, Cotton D. The diagnostic and prognostic value of amniotic fluid white blood cell count, glucose, interleukin-6, and gram stain in patients with preterm labor and intact membranes. Am J Obstet Gynecol 1993;169: 805-16.
¹⁵ Gibbs R S, Cassell G H, Davis J K, St Clair P J. Further studies on genital mycoplasmas in intra-amniotic infection: blood cultures and serologic response. Am J Obstet Gynecol 1986; 154: 717-26.
¹⁶ Weinberger S R, Morris T S, Pawlak M. Recent trends in protein biochip technology. Pharmacogenomics 2000; 1: 395-416.
¹⁷ Petricoin E F, Ardekani A M, Hitt B A, Levine P J, Fusaro V A, Steinberg S M, Mills G B, Simone C, Fishman D A, Kohn E C, Liotta L A. Use of proteomic patterns in serum to identify ovarian cancer. Lancet 2002; 359: 572-7.
¹⁸ Paweletz C P, Trock B, Pennanen M, Tsangaris T, Magnant C, Liotta L A, Petricoin E F 3^{rd}. Proteomic patterns of nipple aspirate fluids obtained by SELDI-TOF: potential for new biomarkers to aid in the diagnosis of breast cancer. Dis. Markers 2001; 17: 301-7.
¹⁹ Davidsson P, Westman-Brinkmalm A, Nilsson C L, Lindbjer M, Paulson L, Andreasen N, Sjogren M, Blennow K. Proteome analysis of cerebrospinal fluid proteins in Alzheimer patients. Neuroreport 2002; 13: 611-5.
²⁰ Buhimschi I A, Buhimschi C S, Weiner C P. Acute versus chronic inflammation: what makes the intrauterine environment “unfriendly to the fetus? From free radicals to proteomics. Am J Reprod Immunol 2003; 49: 319.
²¹ Buhimschi I A, Cristner R, Buhimschi C S, Chaiworapongsa T, Romero R. Proteomic analysis of amniotic fluid for the identification of intra-amniotic inflammation leading to preterm delivery. Am J Obstet Gynecol 2002; 187: S55. Better to cite preliminary patent filing?
²² Locatelli A, Vergani P, Bellini P, Strobelt N, Arreghini A, Ghidini A. Amnioreduction in emergency cerclage with prolapsed membranes: comparison of two methods for reducing the membranes. Am J Perinatol 1999; 16:73-7.
²³ Sherer D M, Salafia C M. Chronic intrauterine bleeding and fetal growth at less than 32 weeks of gestation. Gynecol Obstet Invest 2000; 50: 92-5.
²⁴ Ganz T, Lehrer R I. Antimicrobial peptides of vertebrates. Curr Opin Immunol 1998; 10: 41-4.
²⁵ Kagan B L, Selsted M E, Ganz T, Lehrer R I. Antimicrobial defensin peptides form voltage-dependent ion-permeable channels in planar lipid bilayer membranes. Proc Natl Acad Sci USA 1990; 87: 210-14.
²⁶ Ihi T, Nakazato M, Mukae H, Matsukura S. Elevated concentrations of human neutrophil peptides in plasma, blood, and body fluids from patients with infections. Clin Infect Dis 1997; 25: 1134-40.
²⁷ Panyutich A V, Panyutich E A, Krapivin V A, Baturevich E A, Ganz T. Plasma defensin concentrations are elevated in patients with septicemia or bacterial meningitis. J Lab Clin Med 1993; 122: 202-7.
²⁸ Soong L B, Ganz T, Ellison A, Caughey G H. Purification and characterization of defensins from cystic fibrosis sputum. Inflamm Res 1997; 46: 98-102.
²⁹ Kaiser V, Diamond G. Expression of mammalian defensin genes. J Leukoc Biol 2000; 68: 779-84.
³⁰ Zhang L, Yu W, He T, Caffrey R E, Dalmasso E A, Fu S, Pham T, Mei J, Ho J J, Zhang W, Lopez O, Ho D D. Contribition of human alpha-defensin 1, 2 and 3 ro the anti HIV-1 activity of CD8 antiviral factor. Science 2002; 298: 995-1000.
³¹ Kretsinger R H, Tolbert D, Nakayama S, Pearson W. The EF-hand, homologs and analogs. In Heizmann C W, editor, Novel Calcium Binding Proteins. Fundamentals and Clinical Applications, New York: Springer-Verlag 1991, p 17-37.
³² Rosen T, Kuczynski E, O'Neill L M, Funai E F, Lockwood C J. Plasma levels of thrombin-antithrombin complexes predict preterm premature rupture of the fetal membranes. J Matern Fetal Med. 2001; 10: 297-300.
³³ Elovitz M A, Baron J, Phillippe M. The role of thrombin in preterm parturition. Am J Obstet Gynecol. 2001; 185: 1059-63.
³⁴ Rosen T, Schatz F, Kuczynski E, Lam H, Koo A B, Lockwood C J. Thrombin-enhanced matrix metalloproteinase-1 expression: a mechanism linking placental abruption with premature rupture of the membranes. J Matern Fetal Neonatal Med. 2002; 11: 11-7.
³⁵ Power C, Henry S, Del Bigio M R, Larsen P H, Corbett D, Imai Y, Yong V W, Peeling J. Intracerebral hemorrhage induces macrophage activation and matrix metalloproteinases. Ann Neurol. 2003; 53: 731-42.
³⁶ Buhimschi I A, Buhimschi C S, Weiner C P. Related Protective effect of N-acetylcysteine against fetal death and preterm labor induced by maternal inflammation. Am J Obstet Gynecol 2003; 188: 203-8.
³⁷ Terrone D A, Rinehart B K, Granger J P, Barrilleaux P S, Martin J N Jr, Bennett W A. Interleukin-10 administration and bacterial endotoxin-induced preterm birth in a rat model. Obstet Gynecol 2001; 98: 476-80.
³⁸ Ananth C, Savitz D. Vaginal bleeding and adverse reproductive outcomes: a meta-analysis. Petriatr Perinat Epidemiol 1994; 8: 62-78.
³⁹ Hammouda A. Bleeding in the first two trimesters of pregnancy. Review of 1000 cases. Int Surg 1966; 45: 447-9.
⁴⁰ Chan C C, To W W. Antepartum hemorrhage of unknown origin—what is its clinical significance? Acta Obstet Gynecol Scand 1999; 78: 186-90.
⁴¹ Yang J, Hartmann K E, Savitz D A, Herring A H, Dole N, Olshan A F, Thorp J M. Vaginal bleeding during pregnancy and preterm birth. Am J Epidemiol 2004; 160: 118-125. ⁴² Salafia C M, Lopex-Zeno J A, Sherer D M, Whittington S S Minior V K, Vintzileos A M. Histologic evidence of old intrauterine bleeding is more frequent in prematurity. Am J Obstet Gynecol. 1995 October;173(4):1065-70.
⁴³ MacMullen N J, Dulski L A, Meagher B. Red alert: perinatal hemorrhage. Am J Matern Child Nurs 2005: 30; 46-51
⁴⁴ Nicolini U, Hertogs K, Rodeck C H. Sinusoidal rhythm caused by fetal hemorrhage during fetoscopy. J Perinal Med 1984; 12: 39-42.
⁴⁵ Hankins G D, Rowe J, Quirk J G Jr, Trubey R, Strickland D M. Significance of brown and or green amniotic fluid at the time of second trimester amniocentesis. Obstet Gynecol 1984;64: 353-8.
⁴⁶ Michel B, lgic R, Leray V, Deddish P A, Erdos E G. Removal of Arg141 from the alpha chain of human hemoglobin by carboxypeptidases N and M. Circ Res. 1996 April;78(4):635-42.

1. A method of identifying bleeding within an amniotic environment, comprising:
collecting a sample of the amniotic environment;
determining if a hemoglobin chain is present within the amniotic environment; and
wherein the hemoglobin chain is found to be present within the amniotic environment, determining whether the hemoglobin chain is at least one of a fetal hemoglobin chain, maternal hemoglobin chain and truncated hemoglobin chain.

2. The method of claim 1, further comprising the step of determining old versus recent bleeding by identifying the presence of a truncated alpha hemoglobin chain.

3. The method of claim 1, wherein the identification of bleeding within the amniotic environment is accomplished by analysis of amniotic fluid collected from at least one of an amniocentesis and the vagina.

4. The method of claim 1, wherein the determination of the presence of the hemoglobin chain is by at least one of a mass spectrometic analysis effected via SELDI-TOF or MALDI-TOF methodologies and by a specific immunoglobulin (e.g. ELISA methodology) or a specific chemical reaction based on the structure of the specific marker.

5. The method of claim 1, wherein the hemoglobin chain is indicative of at least one of an incompetent cervix, placental abruption, placenta previa, preterm labor, rupture of a fetal membrane, infection and inflammation.

6. The method of claim 1, further comprising the step of treating the amniotic environment by administering an effective amount of an anti-inflammatory compound, antioxidant compound, or antibiotic.

7. Method of identifying inflammation within an amniotic environment, comprising:
collecting a sample of the amniotic environment supported by an incompetent cervix; and
determining if at least one of a cationic antimicrobial peptide and calcium-binding protein is present within the amniotic environment.

8. The method of claim 7, wherein the cationic antimicrobial peptide is a neutrophil defensin and the calcium-binding protein is a calgranulin.

9. The method of claim 7, wherein the at least one cationic antimicrobial peptide and calcium-binding protein is indicative of at least one of incompetent cervix, placenta previa, preterm labor, rupture of a fetal membrane, infection and inflammation.

10. The method of claim 7, wherein the determination of the presence of the at least one cationic antimicrobial peptide and calcium-binding protein is by at least one of a mass spectrometic analysis effected via SELDI-TOF or MALDI-TOF methodologies and by a specific immunoglobulin (e.g. ELISA methodology) or a specific chemical reaction based on the structure of the specific marker.

11. The method of claim 7, further comprising the step of treating the amniotic environment by administering an effective amount of an anti-inflammatory compound, antioxidant compound, or antibiotic.

12. The method of claim 7, further comprising the step of performing a cerclage procedure upon the incompetent cervix and predicting the success of the cerclage based on the presence of the at least one cationic antimicrobial peptide and calcium-binding protein.

13. A method for treatment of a cervix supporting an amniotic environment, comprising:
determining if a proteomic biomarker is present within the amniotic environment; and
suturing the cervix,
wherein the determination provides a predicitive indication of the clinical success of the cervical suturing in allowing the cervix to continue to support the amniotic environment.

14. The method of claim 13, further comprising the step of determining that the presence of the proteomic biomarker is indicative of at least one of an incompetent cervix, placental abruption, placenta previa, preterm labor, rupture of a fetal membrane, infection and inflammation.

15. The method of claim 14, further comprising the step of treating the inflammation or infection by administering an effective amount of an anti-inflammatory compound, antioxidant compound, or antibiotic.

16. The method of claim 13, wherein the cervix is in a state of pre-term painless cervical dilation.

17. The method of claim 13, wherein the amniotic environment proteomic biomarker is at least one of a cationic antimicrobial peptide, calcium-binding protein, or hemoglobin chain.

18. The method of claim 17, wherein the cationic antimicrobial peptide is a neutrophil defensin and the calcium-binding protein is a calgranulin.

19. The method of claim 13, wherein the determination of the presence of the proteomic biomarker is by at least one of a mass spectrometic analysis effected via SELDI-TOF or MALDI-TOF methodologies and by a specific immunoglobulin (e.g. ELISA methodology) or a specific chemical reaction based on the structure of the specific marker.

20. A method for determining the clinical success of a cerclage performed on a cervix supporting an amniotic environment, comprising:
determining if a proteomic biomarker is present within the amniotic environment supported by the cervix,
wherein the determination provides a predictive indication of the clinical success of the cerclage.

21. The method of claim 20, wherein the cervix is an incompetent cervix.

22. The method of claim 20, wherein the proteomic biomarker is at least one of a cationic antimicrobial peptide, calcium-binding protein, or a hemoglobin chain.

23. The method of claim 22, wherein the cationic antimicrobial peptide is a neutrophil defensin and the calcium-binding protein is a calgranulin.

24. The method of claim 22, further comprising the step of treating the amniotic environment containing at least one of the cationic antimicrobial peptide and calcium-binding protein biomarker with an effective amount of an anti-inflammatory compound, antioxidant compound, or antibiotic.

25. The method of claim 20, wherein the determination of the presence of the proteomic biomarker is by at least one of a mass spectrometic analysis effected via SELDI-TOF or MALDI-TOF methodologies and by a specific immunoglobulin (e.g. ELISA methodology) or a specific chemical reaction based on the structure of the specific marker.
